(19) United States
(12) Patent Application Publication
(10) Pub. No.: US 2023/0075138 A1
(43) Pub. Date: Mar. 9, 2023

(54) METHODS FOR TREATING AND EVALUATING COVID-19 PATIENTS

(71) Applicant: nference, Inc., Cambridge, MA (US)

(72) Inventors: Venkataramanan Soundararajan, Andover, MA (US); Aiveliagaram Venkatakrishnan, Woburn, MA (US)

(21) Appl. No.: 17/863,877

(22) Filed: Jul. 13, 2022

Related U.S. Application Data
(60) Provisional application No. 63/221,667, filed on Jul. 14, 2021.

(51) Int. Cl. C12Q 1/6883; C07K 16/24; C07K 16/28; A61P 31/14; A61K 31/573

(52) U.S. Cl. CPC C12Q 1/6883; C07K 16/248; C07K 16/2866; A61P 31/14; A61K 31/573; C07K 2317/24; C12Q 2600/158; C12Q 2600/106

(57) ABSTRACT

This application relates to methods for assessing patients suffering from coronavirus infection as responders or a non-responders to an immunomodulating therapy, and methods of treating said subjects.

### CROSS-REFERENCE TO RELATED APPLICATIONS

This application claims the benefit of U.S. Provisional Application No. 63/221,667, filed on Jul. 14, 2021, the entire contents of which are incorporated herein in its entirety by this reference.

### BACKGROUND

The COVID-19 pandemic is unquestionably one of the most urgent global health crises of the modern era. Its onset and dissemination around the world has sparked clinical and basic research in efforts to discover interventions which can inhibit or prevent infection or mitigate the clinical course of disease progression. Intensive Care Unit (ICU) admissions and mortality in severe COVID-19 patients are driven by “cytokine storms” and acute respiratory distress syndrome (ARDS). Interim clinical trial results suggest that the corticosteroid dexamethasone displays superior 28-day survival in severe COVID-19 patients requiring ventilation or oxygen. Longitudinal analysis of lab test results pre-and post-corticosteroid administration from 10 hospitalized COVID-19 patients shows ICU duration positively correlates with a change in plasma IL-6 levels. Among a larger cohort of 20 patients with only post-corticosteroid IL-6 measurement, a consistent trend of higher IL-6 levels indicating longer ICU stays is observed.

Infection with SARS-CoV-2 is largely asymptomatic or presents with mild-to-moderate symptoms¹, but can result in progressive respiratory illness leading to acute respiratory distress syndrome (ARDS) in a subset of patients.^{2, 3} Multiple clinical trials are underway to investigate therapies ranging from antivirals (e.g. Lopinavir-Remdesivir) to corticosteroids and targeted immunosuppressive agents (e.g. Dexamethasone, Tocilizumab)⁴. While antiviral drugs, antibiotics, and antimalarials have shown little to no clinical benefit in COVID-19 patients modulation of host inflammatory responses with drugs that inhibit IL-6 signaling (tocilizumab) or stimulate glucocorticoid receptor activity (dexamethasone) have been shown to improve clinical outcomes in critically ill patients.⁶ In line with previous reports of IL-6 as a biomarker of severe disease⁵, some studies examining small patient cohorts have suggested treatment with tocilizumab (anti-IL6 receptor) may improve outcomes in severely ill patients.^{6, 7} The use of IL-6 as a biomarker for disease severity has gained traction during the ongoing COVID-19 pandemic. The high sensitivity plasma IL-6 tests conducted at the Mayo Clinic from 2010 and 2019 show only 274 out of 1463 tests (18%) were above 10 pg/mL (normal range between 0.31-5 pg/mL), compared to 377 out of 537 tests (70%) in the first half of 2020, including both COVID-19 and unrelated IL-6 testing. These estimates suggest that physicians have begun using plasma IL-6 levels to assess COVID-19 disease severity, and further that higher plasma IL-6 levels are likely to continue being reported during the ongoing pandemic relative to the pre-COVID era. In some cases, serial measurements of plasma IL-6 levels may have been used clinically to determine disease progression and treatment efficacy, motivating a thorough examination of the available longitudinal real-world evidence from the Mayo Clinic platform.

A recent interim update from the RECOVERY (Randomised Evaluation of COVid-19 thERapY) trial that is examining larger cohorts of severe COVID-19 patient outcomes revealed the maximal reduction in mortality among patients treated with dexamethasone over other therapies.⁸ In this trial, 2,104 randomized patients received 6 mg of dexamethasone once per day for ten days, compared with 4,321 patients that received standard care alone. Dexamethasone reduced mortality by one-third in ventilated patients (rate ratio 0.65 [95% confidence interval 0.48 to 0.88]; p=0.0003) and by one-fifth in other patients receiving oxygen only (0.80 [0.67 to 0.96]; p=0.0021).⁸

Although corticosteroids have long been utilized clinically for their immunosuppressive and anti-inflammatory capacities, ^{9, 10, 11, 12} the precise mechanisms by which dexamethasone mediates clinical improvement in severe COVID-19 patients are not well understood. Indeed, the success of dexamethasone in treating COVID-19 was somewhat unexpected in light of an early report during this pandemic urging clinicians to not prescribe corticosteroids for lung injury in COVID-19 patients, based on the lack of efficacy as well as elevated risk of adverse events associated with steroid use in the previous SARS and MERS epidemics.¹³ Such ostensibly contradictory evidence and guidance underline the need for an improved mechanistic stratification of corticosteroid efficacy in different subsets of severely ill COVID-19 patients.

### SUMMARY

The present disclosure is based, at least in part, on the longitudinal analysis of real-world data (RWD) of COVID-19 patients and a comprehensive profiling of NR3C1 and triangulate expression-derived insights with human genetic and pharmacologic datasets to nominate putative mechanisms by which dexamethasone results in reduced mortality. Analysis of the single cell RNA-sequence data of bronchoalveolar lavage fluid from severe COVID-19 patients and nearly 2 million human cells from a pan-tissue scan shows alveolar macrophages, smooth muscle cells, and endothelial cells co-express the glucocorticoid receptor NR3C1 and IL-6. In some aspects of the invention disclosed herein, corticosteroids that are NR3C1 agonists may reduce pulmonary and multi-organ inflammation in COVID-19 patients with respiratory failure, by antagonizing IL-6 production in lung macrophages and vasculature. Thus, in some aspects, the invention provides methods of treating a coronavirus infection in a subject in need thereof, comprising a) determining whether the subject is a responder or a non-responder to an immunomodulating therapy by determining the expression level of at least one mRNA of a corticosteroid receptor in a sample obtained from said subject. In preferred embodiments, said expression level is compared with a reference value, wherein the expression level of the corticosteroid receptor relative to the reference value indicates whether the subject will respond to an immunomodulating therapy; and the immunomodulating therapy is administered to the subject whose expression level is indicative of responding to said immunomodulating therapy.

In certain aspects, provided herein are methods of determining whether an immunomodulating therapy is effective for treating a subject having a coronavirus infection. In some embodiments, the method comprises determining the expression level of at least one mRNA of a corticosteroid receptor in a sample obtained from said subject; and comparing the expression level of the corticosteroid receptor with a reference value, wherein a the expression level of the corticosteroid receptor relative to the reference value indicates whether the immunomodulating therapy is effective for treating said subject.

In some aspects, disclosed herein are methods for distinguishing a human subject suffering from coronavirus disease 2019 (CoVID-19) responsive to an immunomodulating therapy from non-responsive subjects, comprising a) obtaining a sample from said subject; b) obtaining a gene expression profile from the sample, wherein the expression profile comprises expression levels for one or more genes; wherein said one or more genes comprise at least one corticosteroid receptor; and c) comparing the gene expression profile of the sample with at least one reference gene expression profile.

In certain aspects of the invention, disclosed herein are methods for monitoring a human subject suffering from CoVID-19 for potential treatment with an immunomodulating therapy. In some embodiments, such methods comprise obtaining a sample from the subject at predetermined intervals. Preferably, a gene expression profile is obtained from the sample(s), wherein the expression profile comprises expression levels for one or more genes; wherein said one or more genes comprises at least one corticosteroid receptor; and the gene expression profile of each sample is compared chronologically, wherein a decrease in corticosteroid receptor expression over time identifies the subject as a responder to an immunomodulating therapy. In some such embodiments, the method further comprising administering an immunomodulating therapy to the subject if the expression of corticosteroid receptor decreases over time. In other embodiments, the method further comprises withholding immunomodulating therapy from the subject if the expression of corticosteroid receptor does not decrease over time.

Unless otherwise defined, all technical and scientific terms used herein have the same meaning as commonly understood by one of ordinary skill in the art to which this disclosure pertains. Preferred methods and materials are described below, although methods and materials similar or equivalent to those described herein can also be used in the practice or testing of the presently disclosed methods and compositions. All publications, patent applications, patents, and other references mentioned herein are incorporated by reference in their entirety.

### BRIEF DESCRIPTION OF THE DRAWINGS

FIG. 1 shows survival curves for hospitalized and ICU patients diagnosed with COVID-19 and treated with one or more of the referenced therapeutic regimen.

FIG. 2 shows the constraining cohort of COVID-19 ICU patients to those who had at least one plasma IL-6 measurement before and after corticosteroid administration. Panel (A) shows longitudinal measurements of plasma IL-6 in hospitalized patients who received non-topical corticosteroids at least one time at or after the diagnosis of COVID-19 via SARS-CoV-2 PCR. Panel (B) shows the relationship between duration of ICU stay and change in plasma IL-6 levels before and after steroid administration. If multiple pre/post-corticosteroid administration levels are available, the mean is used.

FIG. 3 shows the relationship between IL-6 levels and ICU duration for COVID-19 ICU patients who had at least one plasma IL-6 measurement after therapeutic administration. Longitudinal measurements of plasma IL-6 in patients after they received (A) Non-topical corticosteroids (B) Tocilizumab (C) Azithromycin (D) Hydroxychloroquine (E) antivirals at least one time at or after the diagnosis of COVID-19 via SARS-CoV-2 PCR (Patient numbers highlighted here map onto FIG. 13).

FIG. 4 shows the expression profile of NR3C1 across approximately 319,000 non-zero expressing bulk RNA-sequencing samples from the Gene Expression Omnibus. Cells of the immune system are enriched among the highest expressing samples, including both lymphocytes and myeloid cells.

FIG. 5 shows single cell RNA-sequencing expression profile of NR3C1 across approximately 1.9 million individual human-derived cells. Cells of the immune system are enriched among the highest expressing samples, including both lymphocytes and myeloid cells.

FIG. 6 shows expression of NR3C1 in peripheral blood mononuclear cell (PBMC) populations from healthy donors (n=6) COVID-19 patients (n=7) by single cell RNA-sequencing.

FIG. 7 shows expression of NR3C1 in cells from bronchoalveolar lavage fluid (BALF) of COVID-19 patients (n =9; 3 mild, 6 severe) by single cell RNA-sequencing.

FIG. 8 shows the summary of IL-6/NR3C1 co-expression across 1.9 million human cells from single cell RNA-sequencing datasets. Shown are cell and tissue types most highly enriched for detection of both NR3C1 and IL-6 in the same individual cells.

FIG. 9 shows IL-6/NR3C1 co-expression by single cell RNA-sequencing in cells from (A) bronchoalveolar lavage fluid of COVID-19 patients; and (B-D) nasal cavity, respiratory tract, lungs.

FIG. 10 relationship of NR3C1 and IL-6 expression to COVID-19 status and clinical severity. (A) Correlation between IL-6 and NR3C1 expression in the macrophage population found to most robustly co-express these two genes (“Macrophages 4”). Each dot represents the percent of cells from this cluster (i.e. cell population) in which IL-6 and NR3C1 were detected, and dots are colored by COVID-19 status and clinical severity. Values shows are the pearson correlation coefficient (“R”) and corresponding p-value (“p”). (B) Comparison of NR3C1 expression in the “Macrophage 4” cell population from patients with mild versus severe COVID-19.

FIG. 11 shows the distribution of serum or plasma IL-6 measurements for a subset of 700 k patients at the Mayo Clinic taken in 2019 (A) and 2020 (B). Also shown are the enriched ICD codes for the patients in the right tail of the distribution with abnormally high IL-6 levels.

FIG. 12 shows the coexpression of NR3C1 with the two subunits of the GM-CSF receptor (CSF2RA and CSF2RB) in the bronchoalveolar lavage fluid of COVID-19 patients (n=9; 3 mild, 6 severe). Top coexpressing populations, all of which are macrophages, are shown.

FIG. 13 shows the Kaplan-Meier plots for hospitalized COVID-19 patients with at least one IL-6 measurement either 7 days prior to or any time following the first positive PCR test (left) and the subset of those patients who were admitted to the ICU post-COVID diagnosis (right).

FIG. 14 shows the characterization of the overall cohort of n=63 COVIDpos patients in the ICU that received a SARS-CoV-2 positive PCR test (black), a therapeutic treatment (other colors) and plasma IL6 lab test (gray-to-orange gradient).

FIG. 15 shows the characterization of molecular targets of dexamethasone, methylprednisolone, and prednisone. NR3C1 is the highest affinity target for each of these corticosteroids.

FIG. 16 shows the expression of NR3C1 by bulk RNA-sequencing in alveolar macrophages from three independent studies. In one study (Group A, purple, right peaks), alveolar macrophages were found to express high levels of NR3C1, with 13 of 20 samples falling in the top 5% of NR3C1 expression considering all human samples deposited in GEO. In two other studies (Group B, green, left peaks), alveolar macrophages expressed lower but still appreciable levels of NR3C1.

### DETAILED DESCRIPTION

Rapid advances in genomic and transcriptomic technologies over the past decade hold great potential to characterize drug targets at unprecedented levels. The nƒerX® platform Single Cell application was released as a resource to help researchers analyze publicly deposited single cell RNA-sequencing datasets and readily contextualize these expression-derived insights using quantified literature associations¹⁴. this wealth of gene expression data at single cell resolution has been harnessed to profile human tissues and cells based on their expression of ACE2, the putative entry receptor for SARS-CoV-2. ^{14,15,16,17} Notably, although the primary glucocorticoid receptor (NR3C1) has been previously reported to be ubiquitously expressed,^{18,19,20} the global expression profile of this important drug target has not been systematically evaluated across the hundreds of thousands of bulk RNA-seq samples and millions of single cell RNA-sequencing data points which are available.

The present disclosure provides molecular support for the immunomodulatory effects of corticosteroids and the immune cell types which are most likely to be affected by systemic and pulmonary exposure to dexamethasone. Notably, multiple populations of alveolar macrophages in coronavirus-infected patients (e.g., COVID-19 patients) co-express NR3C1 and IL-6 genes, including one population which tends to express lower levels of NR3C1 and higher levels of IL-6 in severely ill patients compared to those with mild disease. Moreover, non-immune cells including endothelial cells and smooth muscle cells are among the cell types which most frequently co-express these genes. Taken together, said disclosure as provided herein, presents molecular evidence that the clinical improvement observed with dexamethasone treatment may be due to NR3C1-mediated antagonism of IL-6 production both systemically and locally in the lungs. Triangulating real world evidence with multi-omics inference over a privacy-preserving ‘precision COVID platform’ thus offered a promising methodology for dissecting the complex immunosuppressive mechanisms underlying corticosteroid therapy.

In certain aspects of the invention, provided herein are methods of treating a coronavirus infection in a subject in need thereof, comprising a) determining whether the subject is a responder or a non-responder to an immunomodulating therapy by determining the expression level of at least one mRNA of a corticosteroid receptor in a sample obtained from said subject. In preferred embodiments, said expression level is compared with a reference value, wherein the expression level of the corticosteroid receptor relative to the reference value indicates whether the subject will respond to an immunomodulating therapy; and the immunomodulating therapy is administered to the subject whose expression level is indicative of responding to said immunomodulating therapy.

In some aspects, provided herein are methods of determining whether an immunomodulating therapy is effective for treating a subject having a coronavirus infection. In some embodiments, the method comprises determining the expression level of at least one mRNA of a corticosteroid receptor in a sample obtained from said subject; and comparing the expression level of the corticosteroid receptor with a reference value, wherein a the expression level of the corticosteroid receptor relative to the reference value indicates whether the immunomodulating therapy is effective for treating said subject.

In some embodiments, the method further comprises determining the expression level of mRNA of IL-6 in the sample obtained from said subject. Preferably the relative expression levels are compared, wherein lower mRNA expression level of the corticosteroid receptor relative to the reference value and concomitant higher IL-6 mRNA expression level relative to an IL-6 reference value indicates that the subject will respond to an immunomodulating therapy. In some such embodiments, the corticosteroid receptor reference value and/or IL-6 reference value indicative of responsiveness to the immunomodulating therapy is representative of one or more subjects who are responsive to the therapy. In other embodiments, a corticosteroid receptor reference value and/or a IL-6 reference value indicative of non-responsiveness to the immunomodulating therapy is representative of one or more subjects who are non-responsive to the therapy. In preferred embodiments, the coronavirus is severe acute respiratory syndrome coronavirus 2 (SARS-CoV2).

In some aspects, disclosed herein are methods for distinguishing a human subject suffering from coronavirus disease 2019 (CoVID-19) responsive to an immunomodulating therapy from non-responsive subjects, comprising a) obtaining a sample from said subject; b) obtaining a gene expression profile from the sample, wherein the expression profile comprises expression levels for one or more genes; wherein said one or more genes comprise at least one corticosteroid receptor; and c) comparing the gene expression profile of the sample with at least one reference gene expression profile. In preferred embodiments, the gene expression profile of the sample is compared with a reference gene expression profile indicative of responsiveness to the therapy obtained from one or more subjects who are responsive to the therapy and/or a reference gene expression profile indicative of non-responsiveness to the therapy obtained from one or more subjects who are non-responsive to the therapy, wherein similarity in expression profiles between the sample and reference profiles indicates sensitivity to the therapy in the subject from whom the sample was obtained, thereby identifying the subject as a responder or non-responder to the therapy.

In certain aspects of the invention, disclosed herein are methods for monitoring a human subject suffering from CoVID-19 for potential treatment with an immunomodulating therapy. In some embodiments, such methods comprise obtaining a sample from the subject at predetermined intervals. Preferably, a gene expression profile is obtained from the sample(s), wherein the expression profile comprises expression levels for one or more genes; wherein said one or more genes comprises at least one corticosteroid receptor; and gene expression profile of each sample is compared chronologically, wherein a decrease in corticosteroid receptor expression over time identifies the subject as a responder to an immunomodulating therapy. In some such embodiments, the method further comprises administering an immunomodulating therapy to the subject if the expression of corticosteroid receptor decreases over time. In other embodiments, the method further comprises withholding immunomodulating therapy from the subject if the expression of corticosteroid receptor does not decrease over time.

In some embodiments, the gene expression profile comprises the expression level of IL-6. In preferred embodiments, a decrease in expression level of the corticosteroid receptor and concomitant increase in IL-6 expression level indicates that the subject will be a responder to an immunomodulating therapy.

In some embodiments the sample comprises, or is derived from, a biological sample from the subject that comprises the cells of the subject, such as a tissue sample or a bodily fluid sample. Such samples include, but are not limited to an organ sample (e.g., lung) or a sample of any fluid present in the body (for example and without limitation, blood, plasma, serum, saliva, synovial fluid, lymph, urine, or cerebrospinal fluid). In preferred embodiments, the sample comprises bronchoalveolar lavage fluid.

In some such embodiments, the sample comprises fibroblasts, epithelial cells, endothelial cells, and smooth muscle cells. In preferred embodiments, the sample comprises peripheral blood mononuclear cells (PBMCs). More preferably, the sample comprises immune cells, such as T cells, B cells, monocytes, natural killer cells, dendritic cells, and macrophages. Most preferably, the sample comprises macrophages, such as alveolar macrophages.

Expression levels and/or gene expression profiles may be obtained from the samples by methods known in the art, e.g., by using RNA-sequencing methods. In some such embodiments, expression levels are determined by bulk RNA-sequencing and/or single cell RNA-sequencing. In preferred embodiments, the expression level is determined by single cell RNA-sequencing, e.g., in immune cells of the sample. In some such embodiments, the immune cells are macrophages and/or T cells.

The immunomodulating therapies contemplated herein may comprise administering a corticosteroid, a corticosteroid receptor agonist, a therapeutic antibody, or any combination thereof. In some embodiments the corticosteroid is a glucocorticoid or a mineralocorticoid. Corticosteroids and agonists contemplated herein include, but are not limited to dexamethasone, prednisone, triamcinolone, methylprednisolone, prednisolone, betamethasone, or hydrocortisone. Preferably, the corticosteroid is dexamethasone. Therapeutic antibodies of the invention (e.g., immunotherapeutic chimeric, humanized, or human monoclonal antibodies, and the like) include anti-cytokine and/or anti-cytokine receptor antibodies. In some such embodiments the therapeutic antibody is an anti-granulocyte macrophage colony-stimulating factor receptor (GM-CSF-R) antibody, such as mavrilimumab. In preferred embodiments, the therapeutic antibody is an anti-IL6 receptor antibody, such as tocilizumab.

The corticosteroid receptors contemplated herein include glucocorticoid receptors and mineralocorticoid receptors. Preferably, the corticosteroid receptor of the invention is a glucocorticoid receptor, also known as NR3C1 (nuclear receptor subfamily 3, group C, member 1).

### EXAMPLES

### Example 1 Methods

Cohort Definition for Patients Receiving IL-6 Measurements and Corticosteroids

All patients who tested positive for SARS-CoV-2 (COVIDₚₒₛ), as determined by at least one positive PCR test, within the Mayo Clinic Health System and were hospitalized, were selected as candidates for further analysis. These COVIDₚₒₛ patients were then filtered by requiring an administration of a systemic corticosteroid at some point during their hospital stay. Corticosteroids included dexamethasone, prednisone, triamcinolone, methylprednisolone, prednisolone, betamethasone, and hydrocortisone. Tocilizumab was also included as a control given it's known effect on IL-6 signaling. In patients who received a systemic corticosteroid, it was required that they underwent plasma IL-6 testing at least once both before and after the first administration of any of the corticosteroid agents listed above. Only 13 patients met these criteria, with 7 patients receiving IL-6 testing before and after methylprednisolone administration, 4 patients receiving IL-6 testing before and after prednisone administration, and 4 patients receiving IL-6 testing before and after hydrocortisone administration. Two patients overlapped drug categories, with one receiving first administrations of both methylprednisolone and hydrocortisone between IL-6 tests and the other receiving first administrations of both prednisone and hydrocortisone between IL-6 tests. In total, six patients received IL-6 testing both before and after any first corticosteroid administration within the timeframe considered (−25 days to +64 days). In addition to IL-6 levels and corticosteroid/tocilizumab administration data, outcomes including death, admission to an ICU, and length of time in ICU, as well as demographic information such as age, sex, and race were extracted.

### Bulk RNA-Sequencing Analysis

Data Accession and Processing:

Datasets were downloaded in raw fastq format and uniformly processed using salmon.

Global Expression Analysis:

To identify highly expressing cells and tissues for a given gene, the following steps were followed:

1. The distribution of gene expression was plotted (in units of transcripts per million, or TPM) across all samples from all studies.

2. The distribution was divided into “High Expression Group” (e.g., cells in top 5% of expressing samples for query gene) and “Low Expression Group” (e.g. cells in bottom 25% of expressing samples for query gene).

3. The number of individual samples from each annotated cell or tissue type falling in the High and Low Expression Groups was counted. Cells and tissues were extracted from sample-level metadata available through the Gene Expression Omnibus (GEO) and other databases including the Genotype-Tissue Expression project (GTEx), The Cancer Genome Atlas (TCGA), and the Cancer Cell Line Encyclopedia (CCLE).

4. Fisher's Exact Test p-value was computed to measure the enrichment of cell type C (or tissue T) among the High Expression Group. Enrichment Scores displayed correspond to-log₁₀(adjusted p-value), where p-values are adjusted using the Benjamini-Hochberg (BH) correction.

### Single Cell RNA-Sequencing Analysis

Data Accession and Processing:

Datasets were downloaded and processed as previously described¹⁴, and processed datasets were made available for investigation upon on-line registration in the nferX Single Cell platform.

Global Expression Analysis:

To identify highly expressing cells and tissues for a given gene, the following steps were followed:

1. The distribution of gene expression was plotted (in units of counts per 10,000, or CP10K) across all single cells from all studies.

2. The distribution was divided into “High Expression Group” (e.g., cells in top 10% of expressing samples for query gene) and “Low Expression Group” (e.g. cells in bottom 90% of expressing samples for query gene).

3. The number of individual cells from each annotated cell population (or tissue) falling in the High and Low Expression Groups was counted.

4. Fisher's Exact Test p-value was computed to measure the enrichment of cell population C (or tissue T) among the High Expression Group. Enrichment Scores displayed correspond to-log₁₀(adjusted p-value), where p-values are adjusted using the Benjamini-Hochberg (BH) correction.

Coexpression Analysis:

For a given set of genes, a single coexpression vector was computed as the geometric mean of CP10K values of all genes in each cell. The geometric mean was used as a coexpression metric as it will only yield a positive value in cells which express all genes in the defined set (i.e. one or more zero values in an individual cell will result in a coexpression value of zero for that cell). As such, all cells with a coexpression value (CP10K_{gm}) greater than zero were considered as “coexpressing cells”, whereas all cells with a CP10K_{gm} values equal to zero were considered as “non-coexpressing cells.” After this coexpression vector was computed, it was treated identically to a gene expression vector for a single gene in the context of the Global Expression or single study-level analyses described above.

Comparison of Patient IL6/ICU Pattern Between Drug Classes:

Four categories of patient IL-6 level/ICU stay length were defined (i.e. the 4 quadrants described in Table 2). For each drug class with at least 10 patients, and for each quadrant with nonzero number of patients, the proportion of patients in the quadrant out of those taking a drug from the class were compared to the proportion of patients in the quadrant out of those not taking a drug from the class. Fisher exact test was performed to compute p-values on these proportion comparisons. A Benjamini-Hochberg adjustment was also applied to these p-values for multiple hypotheses.

### Example 2 Corticosteroids Treatment is Associated with Plasma IL-6 Reductions in a Subset of ICU Patients with COVID-19

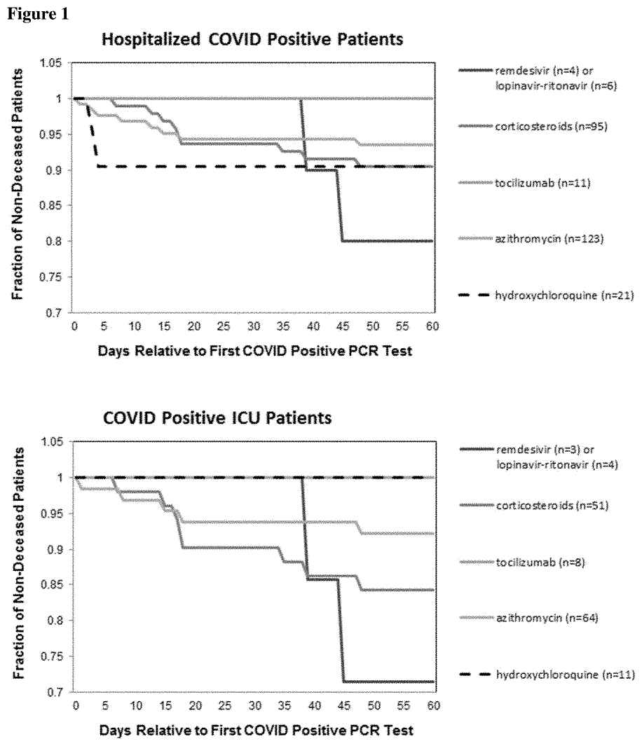

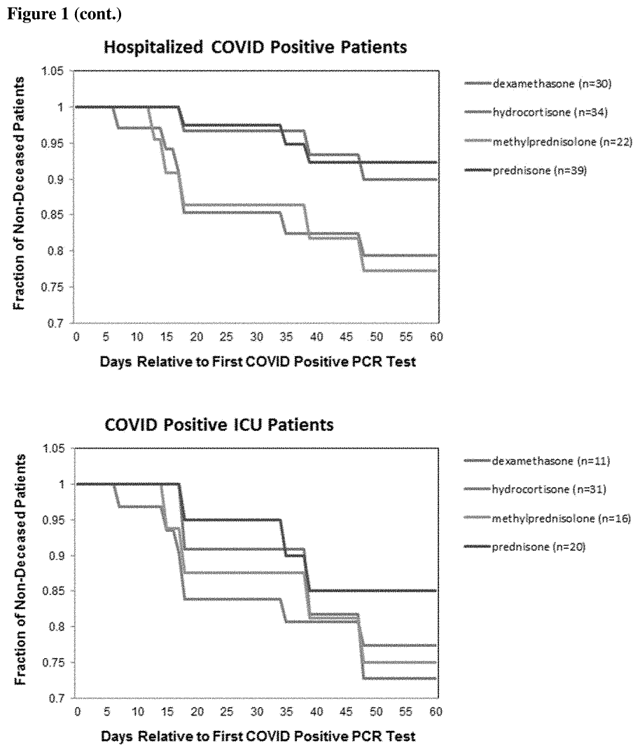

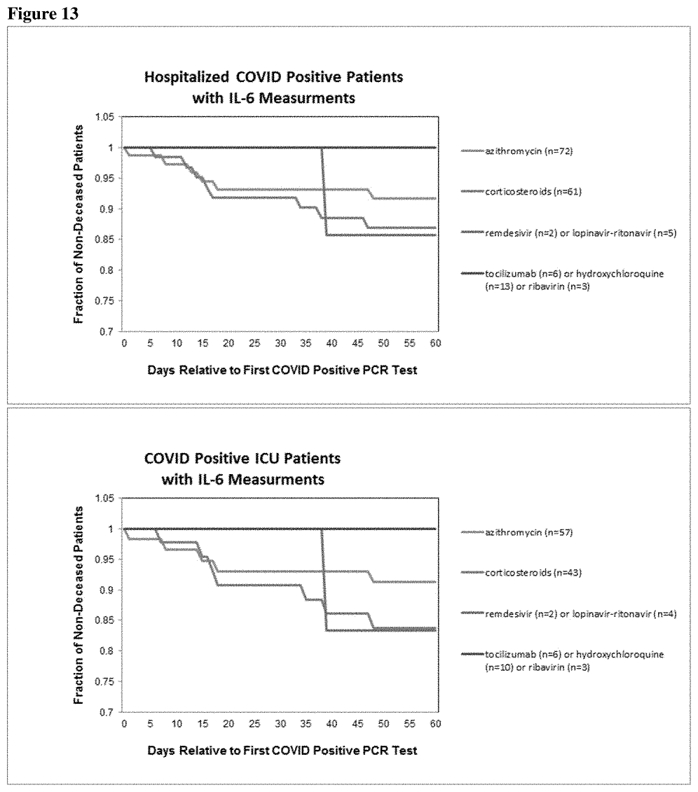

Kaplan-Meier curves were generated for COVID-19 positive patients that were hospitalized at the Mayo Clinic (see FIG. 1). Patient cohorts were defined by each drug or drug class that was administered. Patients receiving multiple drugs of different classes were counted as part of both cohorts. The drugs analyzed included corticosteroids, antivirals (remdesivir, lopinavir-ritonavir), antibacterial azithromycin, hydroxychloroquine, and the IL-6 inhibitor tocilizumab. As shown, all 11 hospitalized patients receiving tocilizumab survived 60 days post diagnosis. Hospitalized cohorts receiving corticosteroids (n=95), azithromycin (n=123), and hydroxychloroquine (n=21) have similar survival rates between 90-95% when considering all hospitalized patients. However, when considering more severe COVID patients requiring ICU admission, this subset of hydroxychloroquine patients (n=11) have a 100% survival rate, the azithromycin cohort (n=64) remains relatively unchanged (92% vs. 93% for all hospitalized patients), and the corticosteroid cohort survival rate decreased from 91% to 84% at 60 days post-diagnosis. In both hospitalized and ICU patient cohorts, antivirals performed most poorly, with the caveat that these cohorts also have the smallest patient counts. Similar trends were also observed in the subset of patients with IL-6 measurements in these cohorts (see FIG. 13)

To further understand the specific effects of the different corticosteroids given to COVID positive patients, survival rates of hospitalized and ICU patients given dexamethasone, hydrocortisone, prednisone and methylprednisolone were also generated. For hospitalized patients, dexamethasone (n=30) and prednisone (n=39) performed similarly (90% and 92% 60-day survival, respectively) and have higher 60-day survival post-diagnosis compared to hydrocortisone (n=34) and methylprednisolone (n=22) (79% and 77% 60-day survival, respectively). While a decrease in survival is observed in the more severe subset of each of these cohorts who are admitted to the ICU, dexamethasone (n=11) shows the most significant decrease in 60-day survival, dropping from 90% to 72%.

### Example 3 Characterization of Hospitalized and ICU Patients Diagnosed with COVID-19 and Their Temporal Therapeutic Regimen

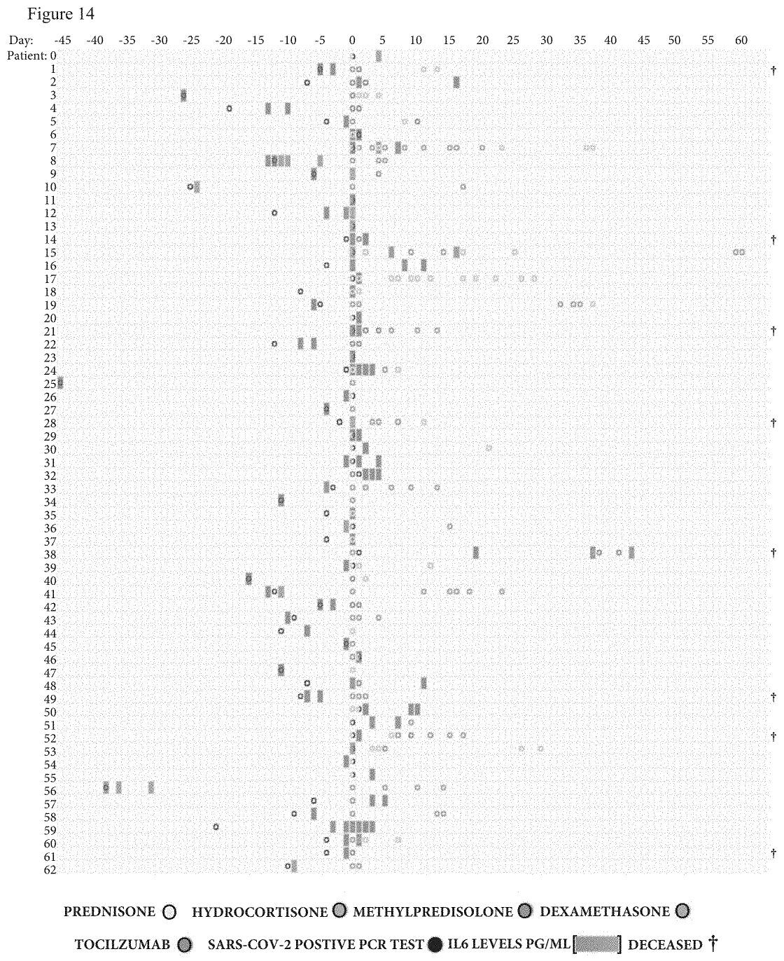

Given the early clinical data showing that both dexamethasone and tocilizumab independently improve outcomes in severely ill COVID-19 patients, the beneficial effect of some anti-inflammatory agents may be in part due to the suppression of IL-6 production. This mechanism would be consistent with the known role of glucocorticoids in reducing IL-6 transcription in macrophages²¹. Plasma IL-6 measurements were taken for 63 hospitalized COVID-19 patients who also received corticosteroids during the course of their care at the Mayo Clinic. The timing of SARS-CoV-2 PCR testing, corticosteroid administration, tocilizumab administration, and IL-6 measurements amongst these patients are highlighted in FIG. 14 (see Methods for details). Of these 63 patients, 10 patients received IL-6 plasma tests after corticosteroid administration and met the following criteria: (1) positive COVID-19 diagnosis, (2) hospitalized in the ICU for COVID-19 related illness, (3) received non-topical corticosteroid therapy during COVID-19 related hospitalization, and (4) underwent plasma IL-6 testing at least one time both before and after the first administration of a corticosteroid. Out of the total number of patients, 4/13 were male and the median age was 65 (interquartile range: 60-78).

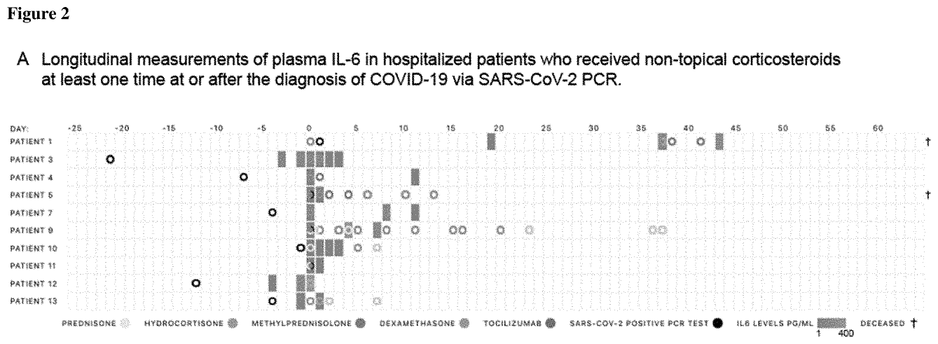

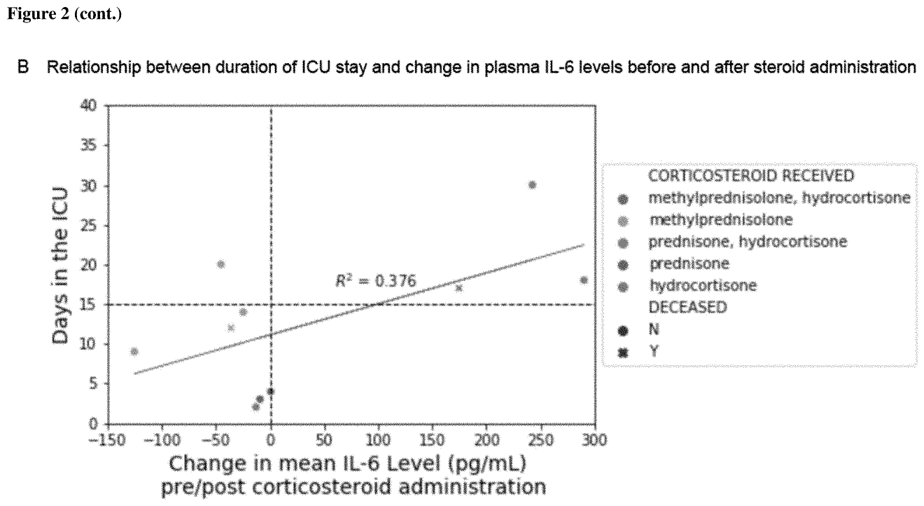

The IL-6 levels remained stable or decreased after steroid administration in 7 of 10 patients, while the remaining 3 patients showed striking increases in IL-6 (see FIG. 2A). Nine of the 10 patients began with pre-corticosteroid IL-6 levels more than twice the upper limit of normal (normal reference range: 0.31-5 pg/mL). Following corticosteroid administration, 5 of the 10 patients returned to mean IL-6 levels less than twice the upper limit of normal (10 pg/mL) and 4 of the 10 patients had IL-6 levels less than 5 pg/mL. Notably, the decreasing/stable IL-6 trends with respect to the administration of steroid therapy were associated with favorable clinical outcomes in COVID-19 for the 10 patients admitted to the ICU, as the duration of ICU stay tended to be shorter in patients with these IL-6 trends compared to those with increasing IL-6 (see FIGS. 2B). Specifically, an ICU duration of 15 days or more was significantly associated with post-steroid increases in IL-6 (p-value =0.03; Table 1).

TABLE 1
Contingency table used to determine if a significant relationship
exists between extended ICU stay and IL-6 measurement
trend pre- and post-corticosteroid administration

| | ICU >= | ICU < | Total |
|---|---|---|---|
| Patient Subset | 15 days | 15 days | (Non-deceassed) |
| IL-6 Stable or Decrease | 6 | 1 | 7 |
| IL-6 Increase | 0 | 3 | 3 |
| Total (Non-deceased) | 6 | 4 | 10 |

Fischer's exact test;
p-value: 0.033

Longitudinal (i.e. pre-and post-corticosteroid) IL-6 testing in similar COVID positive cohorts, with severe symptoms requiring ICU administration, could assist in identifying patients who respond to corticosteroids alone versus those who may require additional anti-inflammatory therapy (e.g. tocilizumab) in combination.

Along these lines, only one of the 10 patients received tocilizumab along with corticosteroid therapy. In this patient, a single dose of tocilizumab was administered on the same day as the first corticosteroid (oral prednisone) administration; the patient subsequently received methylprednisolone. Notably, mean IL-6 levels increased more than 6-fold in this patient following tocilizumab and corticosteroid administration, and the patient had an extended ICU stay (30 days).

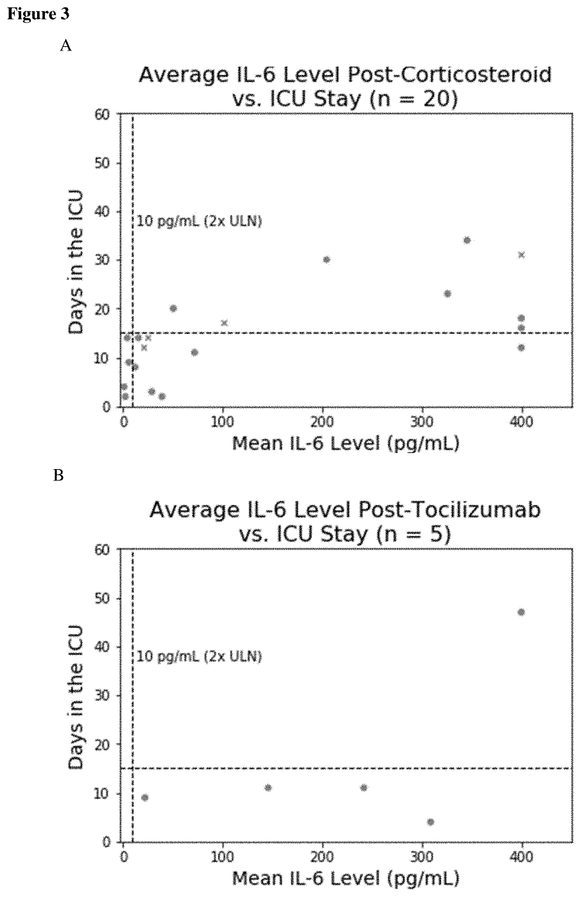

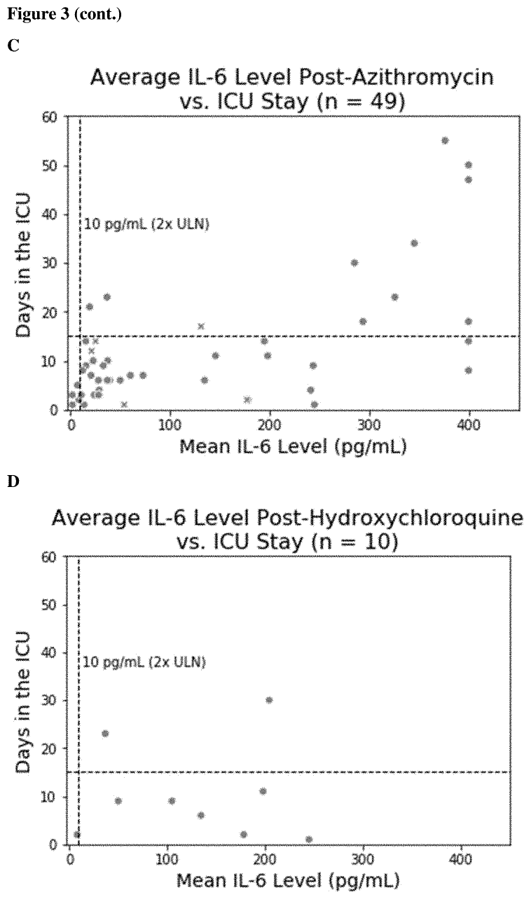

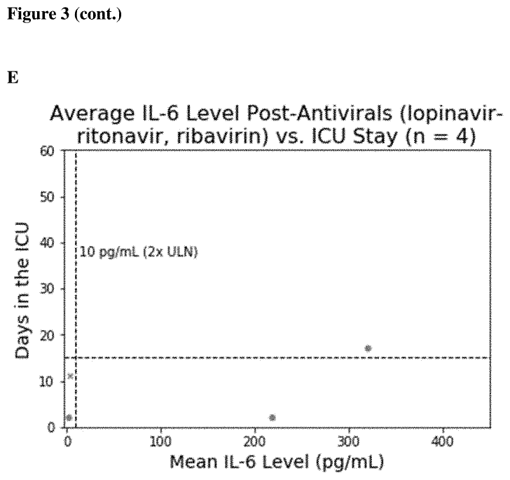

Because only 10 patients met the aforementioned criteria, 10 additional COVID positive, ICU patients who only had IL-6 measured post-corticosteroid administration were also examined. Their IL-6 levels were plotted against the duration of ICU stay (see FIG. 3). Similarly, patients who received tocilizumab (n=5), azithromycin (n=49), hydroxychloroquine (n=10), and antivirals (lopinavir-ritonavir and ribavirin, n=4) were also examined. Patients were grouped into the following four categories based on IL-6 level, ICU status, and mortality:
(Q1) ICU<15 d and IL-6<10 pg/mL,
(Q2) ICU≥15 d or deceased and IL-6<10 pg/mL,
(Q3) ICU<15 d and IL-6≥10 pg/mL, and
(Q4) ICU≥15 d or deceased and IL-6≥10 pg/mL.
   As shown, for patients with IL-6 measured post-corticosteroid administration, half of the patients (10 out of 20) were in Q4, i.e., in the ICU at least 15 days or did not survive and had IL-6 levels greater than 10 pg/mL. As a corollary, corticosteroids have fewer patients (30%, n=20) in Q3 compared to tocilizumab (80%, n=5), azithromycin (61%, n=49), and hydroxychloroquine (60%, n=10). (See Table 2)

TABLE 2
Patient counts and proportions for each quadrant, where deceased
patients were counted with those in the ICU ≥15 days.

| | ICU | | | | | | | | | | |
|---|---|---|---|---|---|---|---|---|---|---|---|
| | Duration | IL-6 | | | | | | | | | |
| | (Days) | (pg/mL) | Corticosteroids | | Tocilizumab | | Azithromycin | | Hydroxychloroquine | | Antivirals |
| Q1 | <15 | <10 | 4 | (0.2) | 0 | (0) | 4 | (0.08) | 2 | (0.2) | 1 (0.25) |
| Q2 | ≥15 OR | <10 | 0 | (0) | 0 | (0) | 0 | (0) | 0 | (0) | 1 (0.25) |
| | deceased | | | | | | | | | | |
| Q3 | <15 | ≥10 | 6 | (0.3) | 4 | (0.8) | 30 | (0.61) | 6 | (0.6) | 1 (0.25) |
| Q4 | ≥15 OR | ≥10 | 10 | (0.5) | 1 | (0.2) | 15 | (0.31) | 2 | (0.2) | 1 (0.25) |
| | deceased | | | | | | | | | | |

| | | | | | |
|---|---|---|---|---|---|
| Total | 20 | 5 | 49 | 10 | 4 |

While not statistically significant due to small cohort sizes (see Table 3), together the results may indicate that patients with high IL-6 levels post-corticosteroids are more likely to have longer ICU durations compared to the other drugs disclosed herein.

TABLE 3
Comparison of quadrants defined in Table 2 to determine if a significant difference
exists between corticosteroids azithromycin, and hydroxychloroquine.

| | | | | | | Proportion | | BH- |
|---|---|---|---|---|---|---|---|---|
| | | | | | | of Group − | | adjusted |
| | Cohort | Other | | Proportion | Proportion | Proportion | p- | p- |
| Cohort | Patients | Patients | Quadrant | of Group | of Other | of Other | value | value |
| Corticosteroid | 20 | 68 | Q1 | 0.20 | 0.10 | 0.10 | 0.26 | 0.47 |
| Corticosteroid | 20 | 68 | Q3 | 0.30 | 0.63 | −0.33 | 0.01 | 0.10 |
| Corticosteroid | 20 | 68 | Q4 | 0.50 | 0.26 | 0.24 | 0.06 | 0.27 |
| Azithromycin | 49 | 39 | Q1 | 0.08 | 0.18 | −0.10 | 0.20 | 0.46 |
| Azithromycin | 49 | 39 | Q3 | 0.63 | 0.46 | 0.17 | 0.13 | 0.40 |
| Azithromycin | 49 | 39 | Q4 | 0.29 | 0.36 | −0.07 | 0.50 | 0.64 |
| Hydroxychloroquine | 10 | 78 | Q1 | 0.20 | 0.12 | 0.08 | 0.61 | 0.68 |
| Hydroxychloroquine | 10 | 78 | Q3 | 0.60 | 0.55 | 0.05 | 1.00 | 1.00 |
| Hydroxychloroquine | 10 | 78 | Q4 | 0.20 | 0.33 | −0.13 | 0.49 | 0.64 |

* Tocilizumab and antivirals were excluded due to low cohort sizes, n = 5 and n = 4, respectively.

Again, this points to the necessity for IL-6 measurement post-corticosteroid administration to monitor if further intervention is needed.

### Example 4 NR3C1 is Strongly Expressed Across Hematopoietic Cell Types Including in Alveolar Macrophages

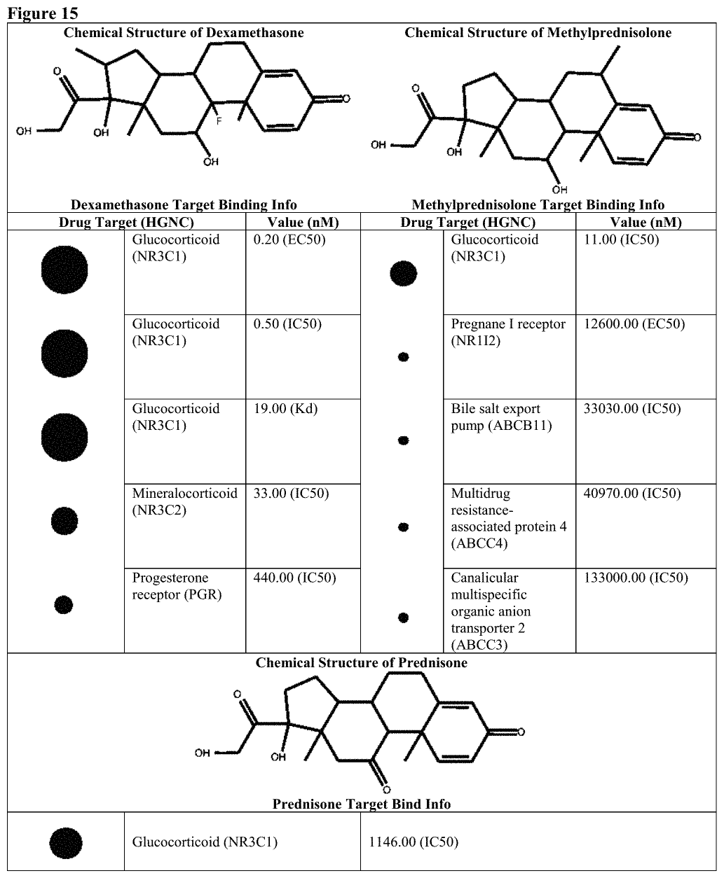

To better understand the mechanisms of potential corticosteroid-induced effects on IL-6 levels, the gene expression of NR3C1—the highest affinity target of dexamethasone, methylprednisolone, and prednisone—systematically profiled across over 450,000 human bulk RNA-sequencing (bulk RNA-seq) samples from more than 10,000 studies (see FIG. 15). Such an analysis is timely and critical to identify potential mechanisms of steroid-induced immunomodulation. Indeed, the immunomodulatory function of corticosteroids has long been appreciated^{9,10}, yet the expression profile of NR3C1 has not been formally established across all human tissues and cell types. The delineation of this profile in both healthy and diseased patients may help us to better understand the cell types which are most critical to mediate the beneficial effects of steroids in severely ill COVID-19 patients.

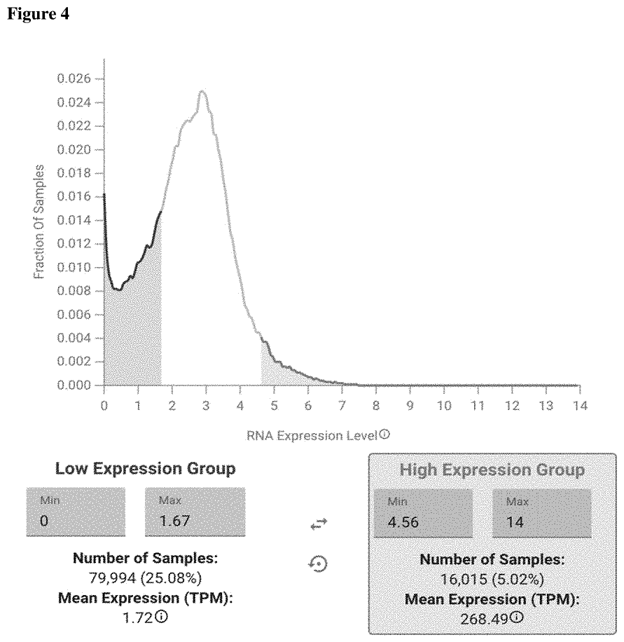

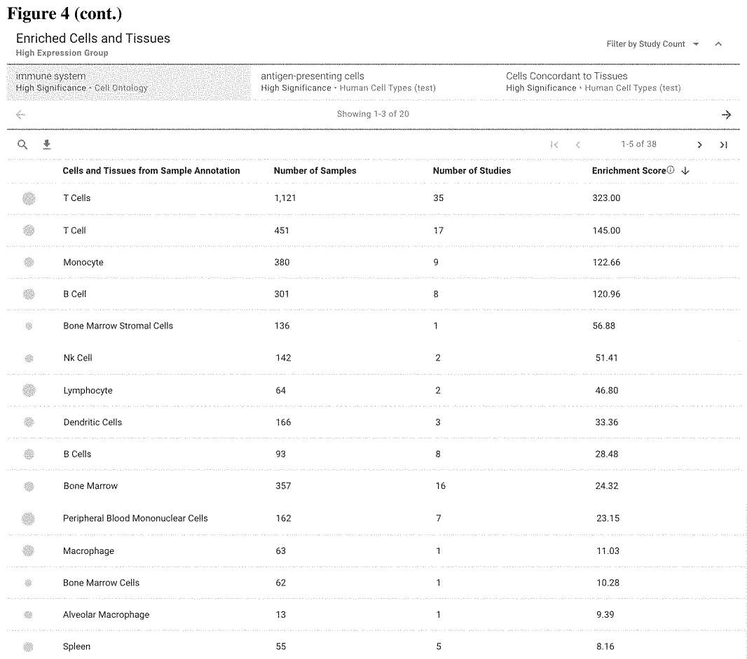

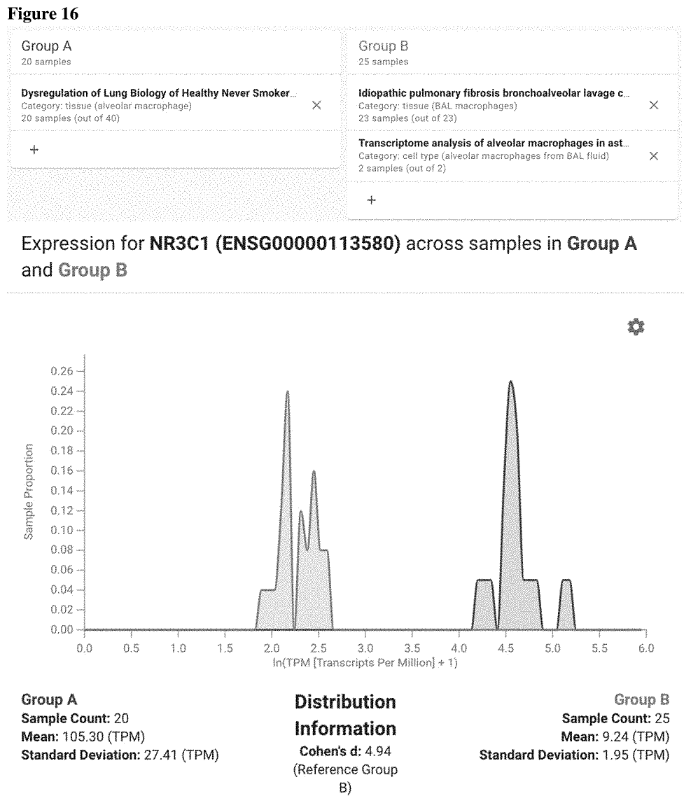

Various immune cells, including T cells, B cells, monocytes, natural killer cells, dendritic cells, and macrophages, were found to be enriched among the samples with the highest (top 5%) of NR3C1 expression (see FIG. 4), consistently with previously reported effects of glucocorticoids on the function of each of these cell types^{22,23,24,25,21}. Interestingly, one study was identified in which alveolar macrophages specifically showed high NR3C1 expression (see FIG. 16; n=20 samples from 10 individuals), and it was found that NR3C1 was consistently expressed at appreciable levels in alveolar macrophages from two other independent studies as well (see FIG. 16; n=25 samples).

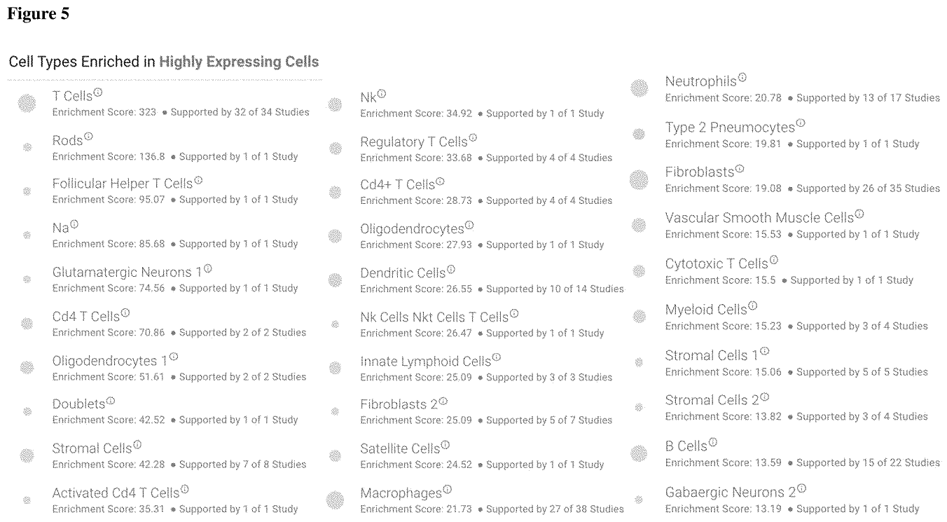

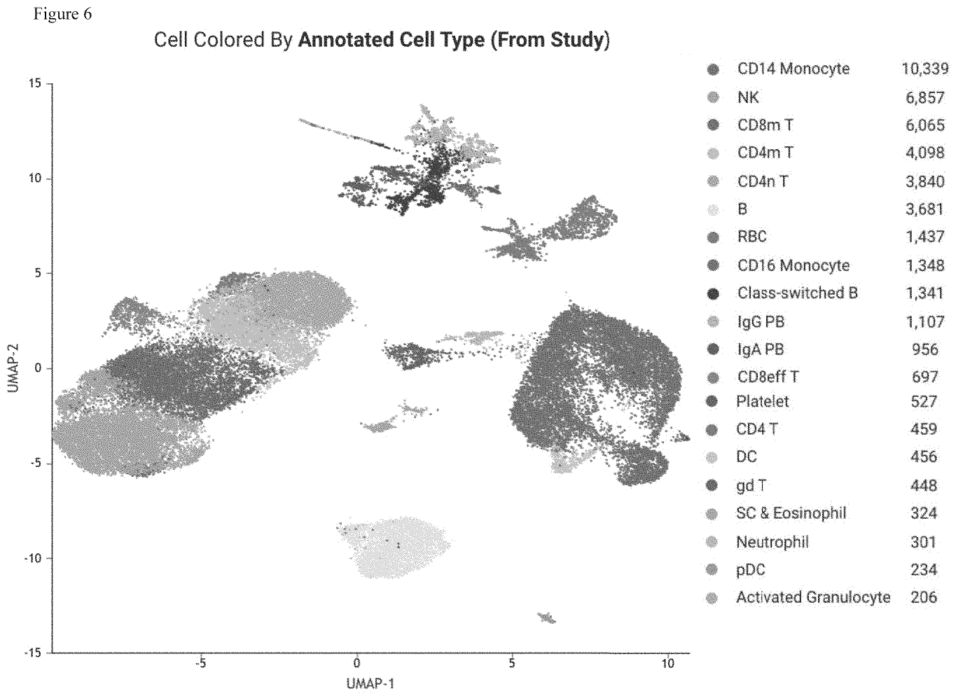

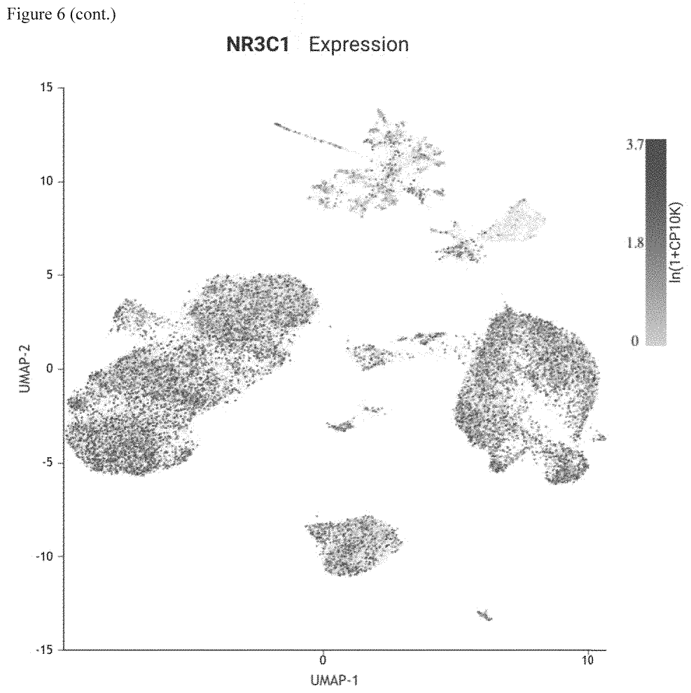

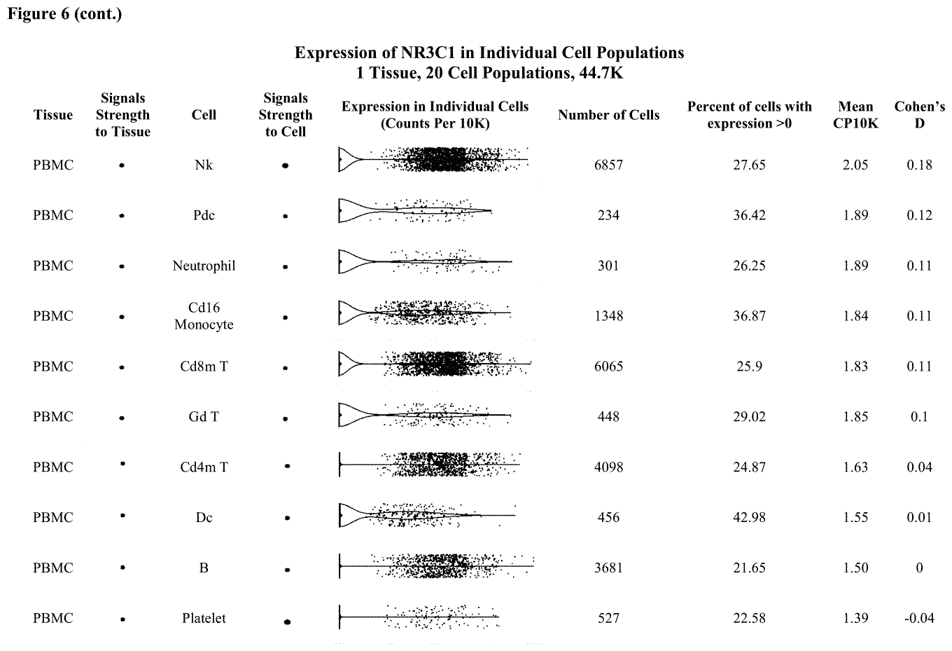

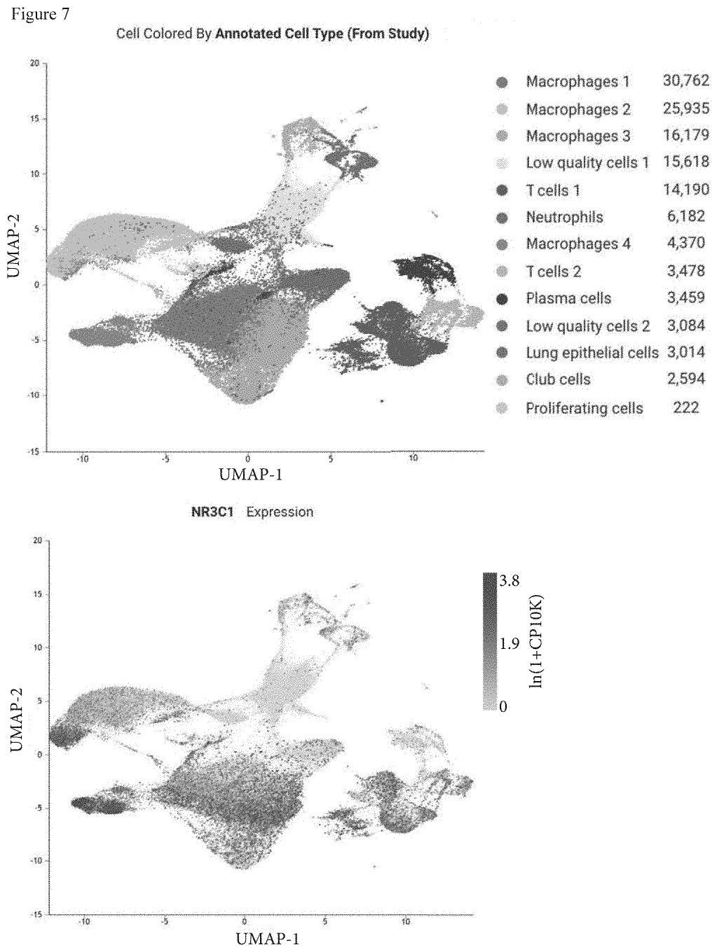

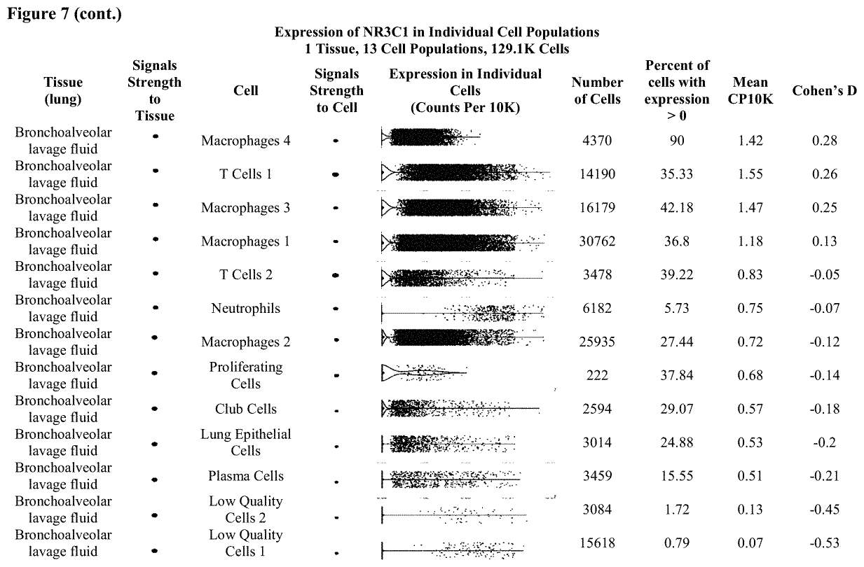

NR3C1 expression was further characterized by single cell RNA-sequencing (single cell RNA-seq) across 1.9 million human cells using the nƒerX® Single Cell platform¹⁴. Consistent with the findings from bulk RNA-seq, various hematopoietic lineages were enriched among the cells with highest expression of NR3C1 (see FIG. 5). Several T cell populations showed particularly strong enrichment among NR3C1-high samples, but evidence was also found for high expression in macrophages, dendritic cells, B cells, and innate lymphoid cells (see FIG. 5). Similarly, in a study of peripheral blood samples from healthy donors (n=6) and COVID-19 patients between days 2 and 16 of symptom onset (n=7), NR3C1 was appreciably expressed in diverse immune cell types including T cells, B cells, dendritic cells, monocytes, and plasmacytoid dendritic cells (see FIG. 6). In bronchoalveolar lavage fluid (BALF) of COVID-19 patients, both macrophages and T cells strongly expressed NR3C1, while plasma cells, neutrophils, and the recovered epithelial populations showed lower levels of NR3C1 expression (see FIG. 7; Single Cell: Severe COVID-19 Patients).

### Example 5 NR3C1 and IL-6 are Coexpressed in Alveolar Macrophages of COVID-19 Patients and Systemically in Endothelial Cells and Smooth Muscle Cells

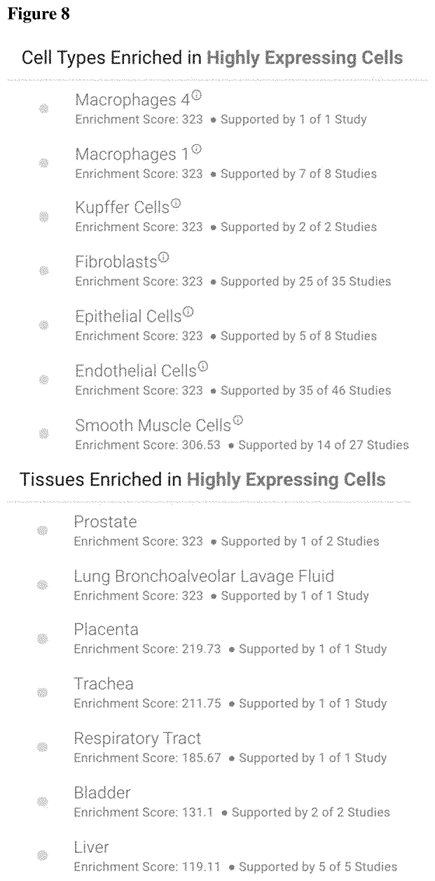

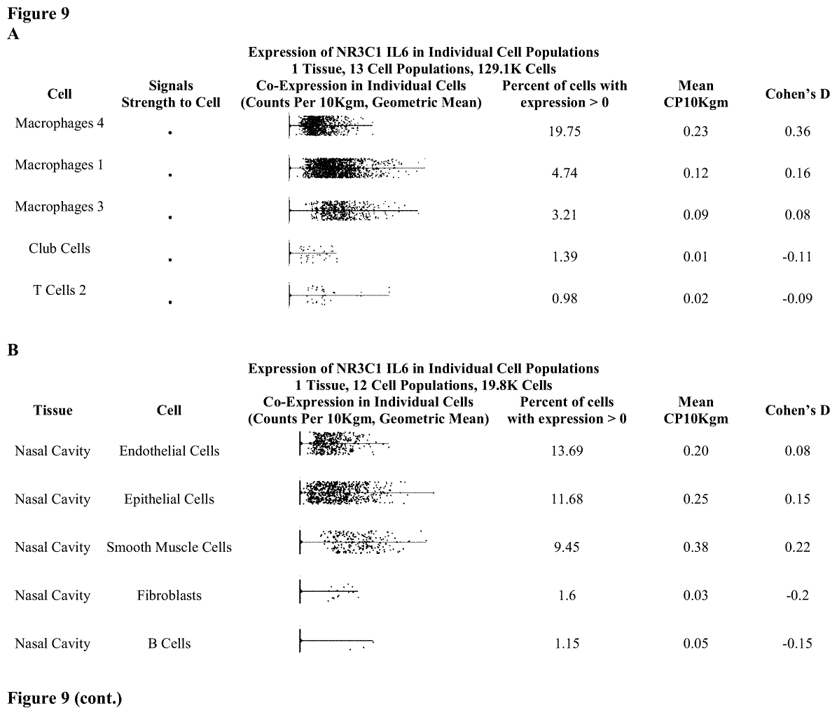

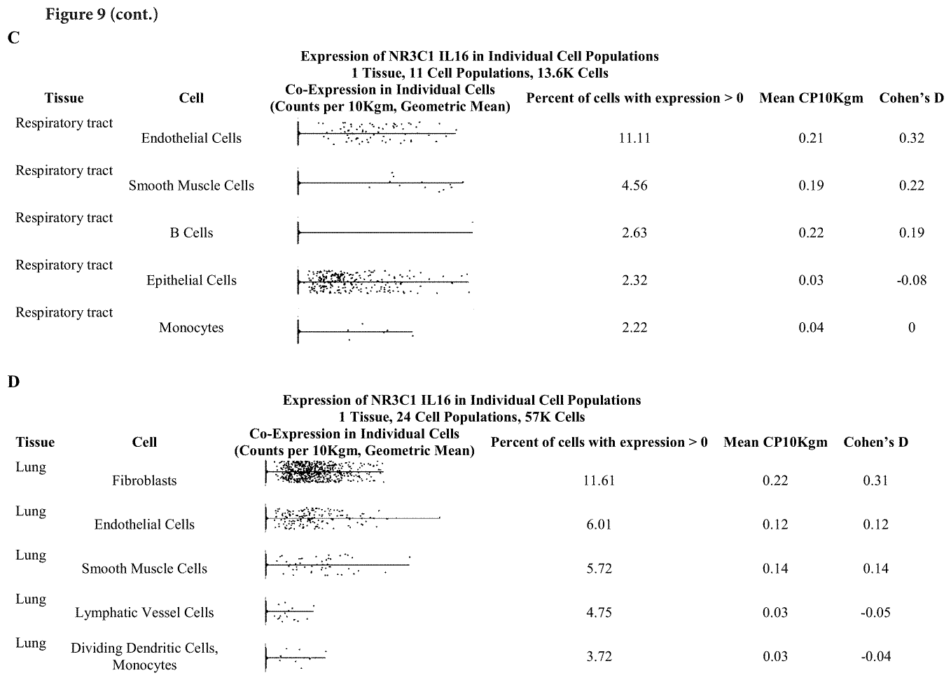

To connect the expression profile to the clinical observation of reduced plasma IL-6 in a subset of patients following corticosteroid administration, co-expression of NR3C1 and IL-6 was specifically evaluated across these same 1.9 million human single cells. Two populations of alveolar macrophages from a study of healthy controls (n=3) and COVID-19 patients (n=9) were among the most strongly enriched cell types for this co-expression (see FIG. 8), with both genes detected in 20% and 5% of these two populations (“Macrophages 4” and “Macrophages 1”, respectively; see FIG. 9A). In contrast to the immune-centric expression profile of NR3C1 alone, several other non-immune cell types showed notable co-expression with IL-6 in both respiratory and non-respiratory tissues including fibroblasts, epithelial cells, endothelial cells, and smooth muscle cells (see FIG. 9B-D). This observation of co-expression in both endothelial cells and smooth muscle cells was particularly interesting given the reports of systemic vascular inflammation in the context of COVID-19²⁶⁻²⁸.

### Example 6 In Severe COVID-19 Patients, NR3C1 is Downregulated in Macrophages that Co-Express IL-6 and NR3C1

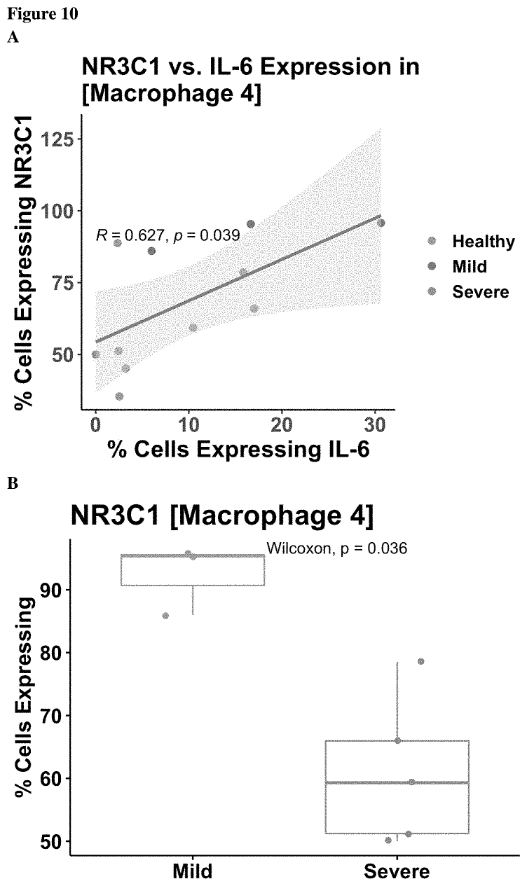

Given that dexamethasone appears to reduce mortality among only severe cases of COVID-19, how NR3C1 and IL-6 co-expression varies between mild and severe disease was investigated. NR3C1 and IL-6 expression levels were assessed in each recovered BALF cell population between cases of mild COVID-19 (n=3) and severe COVID-19 (n=6), along with healthy controls (n=3). Notably, expression of IL-6 and NR3C1 was positively correlated in the strongest co-expressing population (“Macrophages 4” identified above), and further that NR3C1 expression in this cell population was significantly lower in patients with severe disease compared to mild disease (see FIG. 10A-B). This overall direct correlation may reflect a cell-intrinsic mechanism wherein activated inflammatory macrophages are simultaneously primed for homeostatic steroid-mediated immunosuppression. Indeed, such programmed feedback loops are well-established to dampen immune responses, including most notably the immune checkpoint pathways (e.g. PD-1/PD-L1) which restrain excessive T-cell activation upon antigen recognition³⁰. The observation that NR3C1 expression is decreased in severe COVID-19 patients compared to mild COVID-19 patients may reflect a pathologic downregulation of this endogenous immunomodulatory system which can be restored pharmacologically via corticosteroid-mediated agonism of NR3C1. Together with potential broader effects exerted through the various other previously identified NR3C1/IL-6 co-expressing cell types (see FIG. 9), corticosteroid therapy may thereby dampen both local pulmonary and systemic inflammation to reduce the likelihood of patient progression to outright cytokine storm.

### Example 7 Analysis of Dexamethasone and Other Corticosteroids in Subsets of COVID-19 Patients Via NR3C1

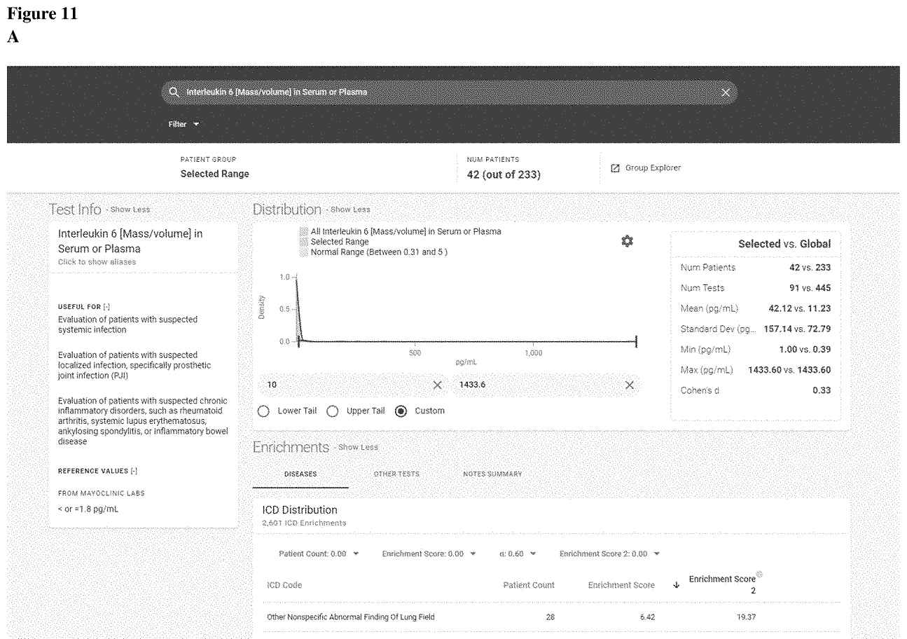

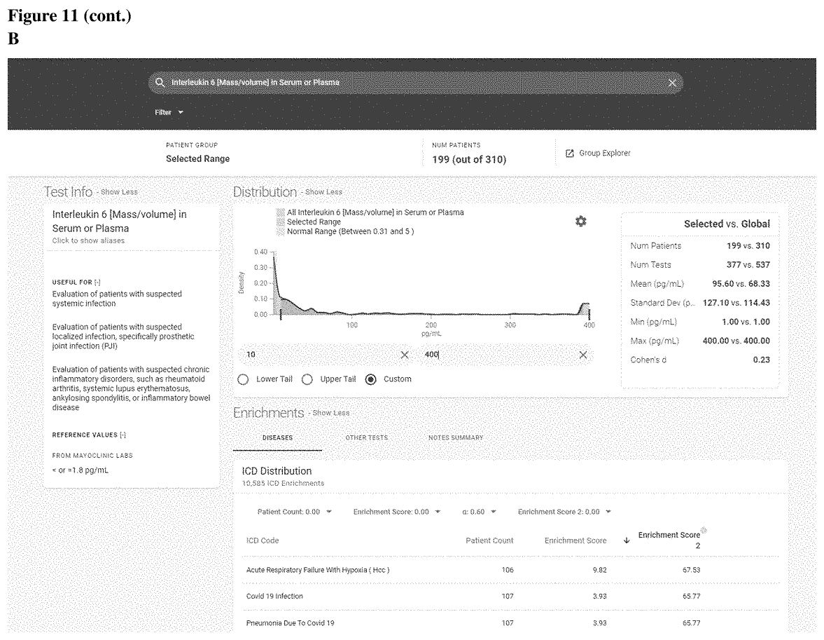

For a subset of 700k patients at the Mayo clinic with rich longitudinal data (i.e. at least 4 encounters, with each being 2-6 weeks apart), it was observed that
(1) the frequency of plasma IL-6 testing increased in the first half of 2020 compared to 2019 (310 patients vs. 233 patients), and
(2) the distribution of observed IL-6 levels in 2020 is shifted towards higher IL-6 levels (mean 68 pg/mL vs. 11 pg/mL) (see FIG. 11).

Further, ICD (International Classification of Diseases) codes enriched among patients with high IL-6 levels in 2019 included “Nonspecific Abnormal Finding of the Lung” whereas those enriched in 2020 include “Acute Respiratory Failure with Hypoxia” and “COVID-19 Infection.” This captured the strong clinical association between high IL-6 levels and severe COVID-19 infection, and suggests the clinical benefit of corticosteroid therapy in critically ill COVID-19 patients is related to an ability to reduce the transcriptional expression of IL-6 and other inflammatory mediators in various cell types.

The integrated analysis of publicly available molecular data and curated electronic health record (EHR) data was prepared to study mechanisms of dexamethasone activity in COVID-19. Despite the long history of corticosteroid use in the clinic, expression of the glucocorticoid receptor (NR3C1) has never been systematically profiled in the modern genomic era to understand the tissues and cell types which are most likely to be directly targeted by systemic steroid therapies. The analysis of gene expression by bulk and single cell RNA-sequencing provided herein suggests that hematopoietic cells are the cells most prominently impacted by dexamethasone. In particular, T cells are the most strongly enriched human cell type for high NR3C1 expression, but other adaptive and innate immune cells are also notably strong expressers.

To analyze the cellular context around the intersection of glucocorticoids and IL-6, the first directed co-expression analysis of NR3C1 and IL-6 across human single cell RNA-sequencing datasets was conducted, totaling almost 2 million cells. The analysis highlighted that while NR3C1 alone is highly expressed in T cells throughout the human body, co-expression with IL-6 is prominently observed in alveolar macrophages of the lung along with various non-immune cells across multiple tissues including endothelial, smooth muscle, epithelial, and stromal cells. This suggests that engagement of the glucocorticoid receptor by dexamethasone in these co-expressing cell types reduces local and systemic IL-6 production, which in turn restores immune homeostasis and mitigates the progression of the COVID-19 associated acute respiratory distress syndrome. The analysis of EHR data from a large academic medical center shows that plasma IL-6 levels are reduced after steroid administration in a subset of critically ill COVID-19 patients. Patients with this steroid-associated IL-6 decline tended to have shorter ICU admissions than patients whose IL-6 levels increased even after steroid administration.

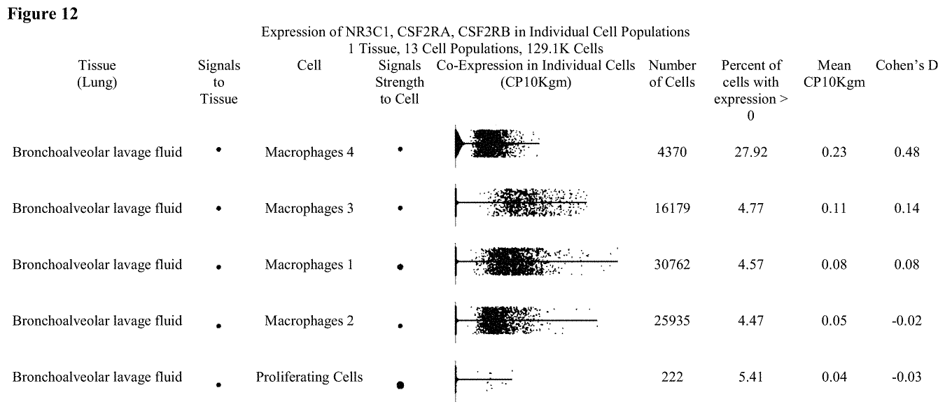

Other inflammatory pathways were also notably activated, and excessively so, in COVID-19 patients. Blockade of these pathways may provide clinical benefit similar to that seen with tocilizumab. For example, early results indicate that blockade of the granulocyte-macrophage colony-stimulating factor (GM-CSF) pathway with the monoclonal antibody mavrilimumab may improve outcomes in severely' several ill patients. GM-CSF is known to orchestrate the activity of various innate immune cells including dendritic cells and macrophages, and the single cell RNA-seq data confirms that NR3C1 is co-expressed with both the alpha and beta subunits of the GM-CSF receptor (CSF2RA and CSF2Rb) in several macrophage populations from the BALF of COVID-19 patients (see FIG. 12). Agonism of the glucocorticoid signaling pathway in these cells may impact their response to GM-CSF and have direct clinical implications regarding the utility of coadministration of corticosteroids with tocilizumab and/or mavrilimumab.

Without being bound by any particular theory, monitoring expression levels of N3CR1 and/or IL-6, or of plasma IL-6 levels after initiation of steroids, may be warranted in clinical practice to determine whether a patient is likely to respond to corticosteroid therapy alone or if they should be considered as candidates for alternative intervention such as tocilizumab.

### References

1. Wagner, T. et al. Augmented Curation of Clinical Notes from a Massive EHR System Reveals Symptoms of Impending COVID-19 Diagnosis. doi:10.1101/2020.04.19.20067660.

2. Zhou, F. et al. Clinical course and risk factors for mortality of adult inpatients with COVID-19 in Wuhan, China: a retrospective cohort study. The Lancet vol. 395 1054-1062 (2020).

3. Goyal, P. et al. Clinical Characteristics of Covid-19 in New York City. N. Engl. J. Med. 382, 2372-2374 (2020).

4. COVID-19 Studies from the World Health Organization Database—ClinicalTrials.gov. https://clinicaltrials.gov/ct2/who_table.

5. Ulhaq, Z. S. & Soraya, G. V. Interleukin-6 as a potential biomarker of COVID-19 progression. Med. Mal. Infect. 50, 382-383 (2020).

6. Kewan, T. et al. Tocilizumab for treatment of patients with severe COVID-19: A retrospective cohort study. EClinicalMedicine 100418 (2020).

7. Xu, X. et al. Effective treatment of severe COVID-19 patients with tocilizumab. Proc. Natl. Acad. Sci. U. S. A. 117, 10970-10975 (2020).

8. Low-cost dexamethasone reduces death by up to one third in hospitalised patients with severe respiratory complications of COVID-19 | University of Oxford. http://www.ox.ac.uk/news/2020-06-16-low-cost-dexamethasone-reduces-death-one-third-hospitalised-patients-severe.

9. Barnes, P. J. Glucocorticosteroids: current and future directions. Br. J. Pharmacol. 163, 29-43 (2011).

10. Coutinho, A. E. & Chapman, K. E. The anti-inflammatory and immunosuppressive effects of glucocorticoids, recent developments and mechanistic insights. Mol. Cell. Endocrinol. 335, 2-13 (2011).

11. Coiffier, B. & Sarkozy, C. Diffuse large B-cell lymphoma: R-CHOP failure-what to do? Hematology Am. Soc. Hematol. Educ. Program 2016, 366-378 (2016).

12. van Staa, T. P. et al. Use of oral corticosteroids in the United Kingdom. QJM 93, 105-111 (2000).

13. Russell, C. D., Millar, J. E. & Baillie, J. K. Clinical evidence does not support corticosteroid treatment for 2019-nCoV lung injury. Lancet 395, 473-475 (2020).

14. Venkatakrishnan, A. J. et al. Knowledge synthesis of 100 million biomedical documents augments the deep expression profiling of coronavirus receptors. Elife 9, (2020).

15. Ziegler, C. G. K. et al. SARS-CoV-2 Receptor ACE2 Is an Interferon-Stimulated Gene in Human Airway Epithelial Cells and Is Detected in Specific Cell Subsets across Tissues. Cell 181, 1016-1035.e19 (2020).

16. Xu, H. et al. High expression of ACE2 receptor of 2019-nCoV on the epithelial cells of oral mucosa. Int. J. Oral Sci. 12, 8 (2020).

17. Li, M.-Y. , Li, L., Zhang, Y. & Wang, X.-S. Expression of the SARS-CoV-2 cell receptor gene ACE2 in a wide variety of human tissues. Infect Dis Poverty 9, 45 (2020).

18. Leventhal, S. M. et al. Uncovering a multitude of human glucocorticoid receptor variants: an expansive survey of a single gene. BMC Genet. 20, 16 (2019).

19. Aranda, C. J. et al. Intestinal epithelial deletion of the glucocorticoid receptor NR3C1 alters expression of inflammatory mediators and barrier function. FASEB J. 33, 14067-14082 (2019).

20. Sefton, C. et al. Elevated Hypothalamic Glucocorticoid Levels Are Associated With Obesity and Hyperphagia in Male Mice. Endocrinology 157, 4257-4265 (2016).

21. Ehrchen, J. M. , Roth, J. & Barczyk-Kahlert, K. More Than Suppression: Glucocorticoid Action on Monocytes and Macrophages. Front. Immunol. 10, 2028 (2019).

22. Cain, D. W. & Cidlowski, J. A. Immune regulation by glucocorticoids. Nat. Rev. Immunol. 17, 233-247 (2017).

23. Olnes, M. J. et al. Effects of Systemically Administered Hydrocortisone on the Human Immunome. Sci. Rep. 6, 23002 (2016).

24. Rinehart, J. J. , Balcerzak, S. P., Sagone, A. L. & LoBuglio, A. F. Effects of corticosteroids on human monocyte function. J. Clin. Invest. 54, 1337-1343 (1974).

25. Oshimi, K., Gonda, N., Sumiya, M. & Kano, S. Effects of corticosteroids on natural killer cell activity in systemic lupus erythematosus. Clin. Exp. Immunol. 40, 83-88 (1980).

26. Varga, Z. et al. Endothelial cell infection and endotheliitis in COVID-19. Lancet 395, 1417-1418 (2020).

27. Teuwen, L.-A., Geldhof, V., Pasut, A. & Carmeliet, P. COVID-19: the vasculature unleashed. Nat. Rev. Immunol. (2020) doi:10.1038/s41577-020-0343-0.

28. Ackermann, M. et al. Pulmonary Vascular Endothelialitis, Thrombosis, and Angiogenesis in Covid-19. N. Engl. J. Med. (2020) doi:10.1056/NEJMoa2015432.

29. Smoak, K. A. & Cidlowski, J. A. Mechanisms of glucocorticoid receptor signaling during inflammation. Mech. Ageing Dev. 125, 697-706 (2004).

30. Sharpe, A. H. Introduction to checkpoint inhibitors and cancer immunotherapy. Immunological Reviews vol. 276 5-8 (2017).

31. Beigel, J. H. et al. Remdesivir for the Treatment of Covid-19—Preliminary Report. N. Engl. J. Med. (2020) doi:10.1056/NEJMoa2007764.

32. Wang, Y. et al. Remdesivir in adults with severe COVID-19: a randomised, double-blind, placebo-controlled, multicentre trial. Lancet 395, 1569-1578 (2020).

33. Boulware, D. R. et al. A Randomized Trial of Hydroxychloroquine as Postexposure Prophylaxis for Covid-19. N. Engl. J. Med. (2020) doi:10.1056/NEJMoa2016638.

34. Horby, P. et al. Effect of Dexamethasone in Hospitalized Patients with COVID-19: Preliminary Report. Infectious Diseases (except HIV/AIDS) (2020) doi:10.1101/2020.06.22.20137273.

### Incorporation by Reference

All publications and patents mentioned herein are hereby incorporated by reference in their entirety as if each individual publication or patent was specifically and individually indicated to be incorporated by reference. In case of conflict, the present application, including any definitions herein, will control.

### Equivalents

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of treating a coronavirus infection in a subject in need thereof, comprising
a) determining whether the subject is a responder or a non-responder to an immunomodulating therapy by
i) determining the expression level of at least one mRNA of a corticosteroid receptor in a sample obtained from said subject;
ii) comparing the expression level determined at step i) with a reference value, wherein the expression level of the corticosteroid receptor relative to the reference value indicates whether the subject will respond to an immunomodulating therapy; and
b) administering the immunomodulating therapy to the subject whose expression level is indicative of responding to said immunomodulating therapy.

2. A method of determining whether an immunomodulating therapy is effective for treating a subject having a coronavirus infection, the method comprising determining the expression level of at least one mRNA of a corticosteroid receptor in a sample obtained from said subject; and comparing the expression level of the corticosteroid receptor with a reference value, wherein the expression level of the corticosteroid receptor relative to the reference value indicates whether the immunomodulating therapy is effective for treating said subject.

3. The method of claim 1, further comprising determining the expression level of mRNA of IL-6 in the sample obtained from said subject.

4. The method of claim 3, wherein lower mRNA expression level of the corticosteroid receptor relative to the reference value and concomitant higher IL-6 mRNA expression level relative to an IL-6 reference value indicates that the subject will respond to an immunomodulating therapy.

5. The method of claim 4, wherein the corticosteroid receptor reference value and/or IL-6 reference value indicative of responsiveness to the immunomodulating therapy is representative of one or more subjects who are responsive to the therapy.

6. The method of claim 4, wherein a corticosteroid receptor reference value and/or a IL-6 reference value indicative of non-responsiveness to the immunomodulating therapy is representative of one or more subjects who are non-responsive to the therapy.

7. The method of claim 1, wherein the coronavirus is severe acute respiratory syndrome coronavirus 2 (SARS-CoV2).

8. A method for distinguishing a human subject suffering from coronavirus disease 2019 (CoVID-19) responsive to an immunomodulating therapy from non-responsive subjects, comprising:
a) obtaining a sample from said subject;
b) obtaining a gene expression profile from the sample, wherein the expression profile comprises expression levels for one or more genes; wherein said one or more genes comprise at least one corticosteroid receptor; and
c) comparing the gene expression profile of the sample with at least one reference gene expression profile.

9. The method of claim 8, wherein the gene expression profile of the sample is compared with a reference gene expression profile indicative of responsiveness to the therapy obtained from one or more subjects who are responsive to the therapy and/or a reference gene expression profile indicative of non-responsiveness to the therapy obtained from one or more subjects who are non-responsive to the therapy, wherein similarity in expression profiles between the sample and reference profiles indicates sensitivity to the therapy in the subject from whom the sample was obtained, thereby identifying the subject as a responder or non-responder to the therapy.

10. A method for monitoring a human subject suffering from CoVID-19 for potential treatment with an immunomodulating therapy, comprising obtaining a sample from the subject at predetermined intervals;
a) obtaining a gene expression profile from the sample, wherein the expression profile comprises expression levels for one or more genes; wherein said one or more genes comprises at least one corticosteroid receptor;
b) comparing the gene expression profile of each sample chronologically, wherein a decrease in corticosteroid receptor expression over time identifies the subject as a responder to an immunomodulating therapy; and
c) administering an immunomodulating therapy if the expression of corticosteroid receptor decreases over time, said treatment comprising administering an immunomodulating therapy to the subject.

11. (canceled)

12. The method of claim 10, further comprising withholding immunomodulating therapy from the subject if the expression of corticosteroid receptor does not decrease over time.

13. The method of claim 8, wherein the gene expression profile comprises the expression level of IL-6, wherein a decrease in expression level of the corticosteroid receptor and concomitant increase in IL-6 expression level indicates that the subject will be a responder to an immunomodulating therapy.

14. (canceled)

15. The method of claim 1, wherein the sample comprises PBMCs or bronchoalveolar lavage fluid.

16. (canceled)

17. The method of claim 1, wherein the expression level is determined by bulk RNA-sequencing, by single cell RNA-sequencing, or any combination thereof.

18. (canceled)

19. The method of claim 1, wherein the expression level is determined in immune cells of the sample, wherein the immune cells are macrophages and/or T cells.

20. (canceled)

21. The method of claim 1, wherein the immunomodulating therapy comprises administering a corticosteroid, a corticosteroid receptor agonist, a therapeutic antibody, or any combination thereof.

22. The method of claim 21, wherein the corticosteroid is a glucocorticoid.

23. The method of claim 21, wherein the corticosteroid is dexamethasone, prednisone, triamcinolone, methylprednisolone, prednisolone, betamethasone, or hydrocortisone.

24. (canceled)

25. The method of claim 21, wherein the therapeutic antibody is an anti-IL6 and/or an anti-granulocyte macrophage colony-stimulating factor receptor (GM-CSF-R) antibody.

26-27. (canceled)

28. The method of claim 1, wherein the corticosteroid receptor is nuclear receptor subfamily 3, group C, member 1 (NR3C 1).
